(19) United States
(12) Patent Application Publication
(10) Pub. No.: US 2025/0213839 A1
(43) Pub. Date: Jul. 3, 2025

(54) Microneedle Tattoo Patches and Methods

(71) Applicant: Georgia Tech Research Corporation, Atlanta, GA (US)

(72) Inventors: Mark R. Prausnitz, Atlanta, GA (US); Song Li, Atlanta, GA (US)

(21) Appl. No.: 18/848,775

(22) PCT Filed: Mar. 22, 2023
(86) PCT No.: PCT/US2023/015982

§ 371 Date: Sep. 19, 2024

Related U.S. Application Data
(60) Provisional application No. 63/322,636, filed on Mar. 22, 2022.

(51) Int. Cl. A61M 37/00; A61Q 1/02

(52) U.S. Cl. CPC A61M 37/0076; A61Q 1/025; A61M 2037/0023; A61M 2037/0046; A61M 2037/0053; A61M 2037/0061; A61M 2250/00

(57) ABSTRACT

A microneedle patch for creating a tattoo image on skin, wherein the microneedle patch includes a backing layer, and an array of microneedles extending from the backing layer, wherein the microneedles each include a distal tip portion that has a tattoo substance, wherein the microneedles are configured to be inserted into a subject's skin and to release the tattoo substance in the skin, thereby creating a tattoo image in the skin that is visible to the human eye. Each microneedle in the array of microneedles may correspond to a dot, or a pixel, of the tattoo image or a portion thereof.

### CROSS-REFERENCE TO RELATED APPLICATIONS

This application claims priority to U.S. Provisional Application No. 63/322,636, filed on Mar. 22, 2022, which is incorporated herein by reference.

### BACKGROUND

Tattoos have been used in human societies and cultures around the world for millennia as a form of body modification that provides art, information, spiritual meaning or other attributes. Tattoos are in widespread use, both for medical or veterinary applications as well as for ornamental, self-expression, or cosmetic purposes. For example, medical tattoos are used to communicate chronic or life-threatening medical conditions like diabetes to emergency personnel, to identify body locations for repeated therapeutic treatment such as radiotherapy, or to treat cosmetic outcomes of medical conditions, such as dermatographic color correction of vitiligo and simulating areola in nipple-areola reconstruction therapy. Animals are tattooed to indicate sterilization status or for individual identification and/or population monitoring of free-roaming animals.

Tattoos are typically made by injecting pigments or inks into the skin using needles or lancets. Modern tattoo machines use electromagnetic force to move the needles in and out of the skin repeatedly at high frequency up to several thousand times per minute to deposit material in the skin, and the needles can penetrate the skin to a depth from several hundred micrometers up to around 2 mm. The liquid tattoo ink is guided via the needle and deposited in the skin. Although many different tattooing methods have been developed throughout human history, the basic principle of introducing colored particles into the skin by needle puncture is largely unchanged.

Adverse effects of tattooing have been reported with incidence as high as 31% and 68% describing skin complications after receiving a tattoo, most commonly pruritus and/or bleeding. There is also a risk of infection, which is primarily bacterial and localized to the skin, with an incidence of up to 5% of tattoo recipients.

Accordingly, there is a need to provide new and improved methods and systems for applying tattoos.

### BRIEF SUMMARY

In one aspect, a microneedle patch is provided for creating a tattoo image on skin, wherein the microneedle patch includes a backing layer, and an array of microneedles extending from the backing layer, wherein the microneedles each include a distal tip portion that has a tattoo substance, wherein the microneedles are configured to be inserted into a subject's skin and to release the tattoo substance in the skin, thereby creating a tattoo image in the skin that is visible to the human eye. Each microneedle in the array of microneedles may correspond to a dot, or a pixel, of the tattoo image or a portion thereof.

In another aspect, a kit is provided for creating a tattoo image comprising: two or more microneedle patches, each patch comprising: a backing layer; and an array of microneedles extending from the backing layer; wherein each microneedle of the array of microneedles comprises a distal tip portion comprising a tattoo substance, wherein the microneedles of each of the two or more microneedle patches are configured to be inserted into skin to form a tattoo image. In particular embodiments, the two or more microneedle patches are configured to cooperate to produce a composite tattoo resulting from the tattoo images from each microneedle patch being selectively positioned relative to one another, such as adjacent to one another and/or overlapping one another.

In still another aspect, methods of making a tattoo microneedle patch are provided. The method may include casting a first composition in a mold having a cavity defining an array of microneedles, wherein the first composition comprises a tattoo substance; and then solidifying the first composition in the mold to form the array of microneedles, or at least a distal tip portion of the microneedles, in the mold, wherein each microneedle in the array of microneedles corresponds to a dot, or a pixel, of a predefined tattoo image or a portion thereof.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may or may not be present in various embodiments. Elements and/or components are not necessarily drawn to scale.

FIG. 1 is a perspective view of microneedle patch, which comprises an array of microneedles, according to one or more embodiments of the present disclosure.

FIG. 2A is a microphotograph showing a perspective view of an array of tattoo microneedles, according to one or more embodiments of the present disclosure.

FIG. 2B is a microphotograph showing a close-up of the tattoo microneedles shown in FIG. 2A.

FIG. 3A is a microphotograph showing a perspective view of an array of remaining proximal portions of tattoo microneedles after the distal tip portions of the microneedles have dissolved, according to one or more embodiments of the present disclosure.

FIG. 3B is a microphotograph showing a close-up of the remaining proximal portions of tattoo microneedles shown in FIG. 3A.

FIGS. 4A-4B are plan views of microneedle molds in the shape of a heart and a star, respectively, according to one or more embodiments of the present disclosure.

FIGS. 4C-4D are perspective views of tattoo microneedle arrays made using the molds illustrated in FIGS. 4A-4B, respectively, according to one or more embodiments of the present disclosure.

FIGS. 4E-4F are plan views of tattoo images in an animal model skin made using the tattoo microneedle arrays shown in FIGS. 4C-4D, respectively, according to one or more embodiments of the present disclosure.

FIGS. 5A-5C are plan views of microneedle molds in the shapes of medical indicators, according to one or more embodiments of the present disclosure.

FIGS. 5D-5F are perspective views of tattoo microneedle arrays made using the molds illustrated in FIGS. 5A-5C, respectively, according to one or more embodiments of the present disclosure.

FIGS. 5G-5I are plan views of tattoo images in animal model skin made using the tattoo microneedle arrays shown in FIGS. 5D-5F, respectively, according to one or more embodiments of the present disclosure.

FIGS. 6A-6B are perspective views tattoo microneedle arrays utilizing light responsive pigment, according to one or more embodiments of the present disclosure.

FIGS. 6C-6E are plan views of tattoo images in animal model skin made using the tattoo microneedle arrays shown in FIGS. 6A-6B viewed under daylight, darkness and UV light, and daylight and UV light, respectively, according to one or more embodiments of the present disclosure.

FIGS. 7A-7B are perspective views of tattoo microneedle arrays utilizing thermally responsive pigment, according to one or more embodiments of the present disclosure.

FIGS. 7C-7D are plan views of the tattoo images in animal model skin made using the tattoo microneedle arrays shown in FIGS. 7A-7B in heated and unheated states, respectively, according to one or more embodiments of the present disclosure.

FIG. 8A illustrates a QR code pattern used as the basis for a tattoo microneedle array, according to one or more embodiments of the present disclosure.

FIG. 8B is a perspective view of a tattoo microneedle array depicting the QR code of FIG. 8A, according to one or more embodiments of the present disclosure.

FIG. 8C is a plan view of the tattoo image in animal model skin made using the tattoo microneedle array shown in FIG. 8B, according to one or more embodiments of the present disclosure.

### DETAILED DESCRIPTION

Tattoo microneedle arrays and patches have been developed wherein the array of microneedles is (i) configured, e.g., has the geometry and mechanical properties, to be inserted into skin (or other mammalian tissue), and (2) configured, e.g., has a suitable composition, to release a tattoo substance in the skin to create the tattoo image in the skin.

In particular, the tattoo image is one that is directly visible to the human eye, i.e., the “naked” eye (which may include the use of conventional eyeglasses or contact lenses). An image that cannot be seen in ambient light but that fluoresces only in near infrared (NIR) wavelengths would not be considered “visible to the human eye” as used herein.

The feasibility of microneedle patches to administer tattoos to the skin for a variety of medical and other applications has been demonstrated. The microneedle patches simplify the tattooing process by reducing the need for technical and artistic skill to administer tattoos and increase acceptability of the process by tattoo recipients by reducing time and eliminating pain for improve tolerability. Tattoo microneedle patches described herein advantageously may be provided as single-use, sterile, skin patches that generate no biohazardous sharps waste. The microneedle patch tattoos also may be capable of being responsive to environmental cues, such as light or temperature, which could be used to monitor physiological processes in situ in the body for medical, research or other purposes. The microneedle patches also enable administration of complex tattoo patterns like QR codes.

One or more microneedle patches are described herein for creating a tattoo image on skin. The microneedle patch generally includes a backing layer, and an array of microneedles extending from the backing layer, wherein the microneedles each have a distal tip portion which comprises a tattoo substance, and the microneedles are configured to be inserted into a subject's skin, and wherein the microneedles are configured to release the tattoo substance in the skin, thereby creating the tattoo image in the skin.

Each microneedle in the array of microneedles may correspond to a dot, or a pixel, of the tattoo image or a portion thereof. This may be particularly advantageous to aid in creating tattoo images of a size and/or complexity needing the application of two or more microneedle patches to complete.

In some embodiments, the subject is a human, and the tattoo image can be placed on essentially any area of the person's skin which the person desires to have the tattoo image. In some other embodiments, the subject is a domesticated animal, such as a pet (e.g., a dog or cat) or livestock (e.g., cattle, swine, sheep, or horse). For domesticated animals, the tattoo image may include identifying information, such as the owner of the animal and/or the animal's name or tracking number, or medical status information, such as vaccination or sterilization status. In some embodiments, the tattoo image includes medical/veterinary alert or medical/veterinary record information selected from vaccination status, allergies, blood type, medical conditions (e.g., asthma, epilepsy, diabetes), medications (e.g., blood thinner, vaccination, sterilization), medical devices (e.g., pacemaker), do-not-resuscitate orders, and contact information, and/or encoded personal health information.

In one specific example, a tattoo image, providing sterilization status, is placed in the underside of an ear or on the belly near the incision site from sterilization surgery of the dog, cat, or other animal. In another specific example, a tattoo image, providing medical alert status, is placed about the hand, wrist, or arm of a human, so that it may be prominently displayed or at least readily visible, for example, to an emergency medical worker. Because these medical tattoo patches can be easily administered and read, they can enable patients to provide valuable medical information to health care personnel, such as first responders in an emergency involving, for example, extensive blood loss or a severe hypoglycemic event where the patient may be unresponsive or incoherent, potentially replacing the need for conventional medical alert bracelets.

The tattoo image may include any symbols, numbers, letters and other medical/decorative images applied to the skin. The tattoo image may be pictorial in nature, depicting a representative, abstract or other image. The tattoo image may involve geometric patterns, representations of real or imagined, living or inanimate physical objects. The tattoo image may serve the function of make-up to enhance appearance of the face or other body part, such as eye liner, eyebrow, lipstick, scalp micropigmentation, etc. The tattoo image may include encoded information, i.e., information that has been converted into a particular visual form, such as a machine-readable form, for example a bar code or QR code. The encoded information may be a URL, for instance. Compared to simpler tattoo patterns, creating a QR code with a microneedle patch has the advantage of storing much more information or providing a link to essentially unlimited information on the cloud.

The tattoo image produced by the microneedle patch may be substantially permanent or temporary, for example, depending on the formulation of the tattoo substance/microneedle, and the amount and location of the tattoo substance delivered into the skin. Temporary tattoo images may be particularly desirable for some cosmetic or costume-related tattoos.

One benefit of the tattoo microneedle patches described herein is that they may be configured for a person to self-administer the tattoo image. The present microneedle patches also advantageously are essentially a dry system, in that they do not require the user to handle or inject liquid inks or other liquid forms of tattoo substances, as with conventional tattooing.

### Microneedle Patches

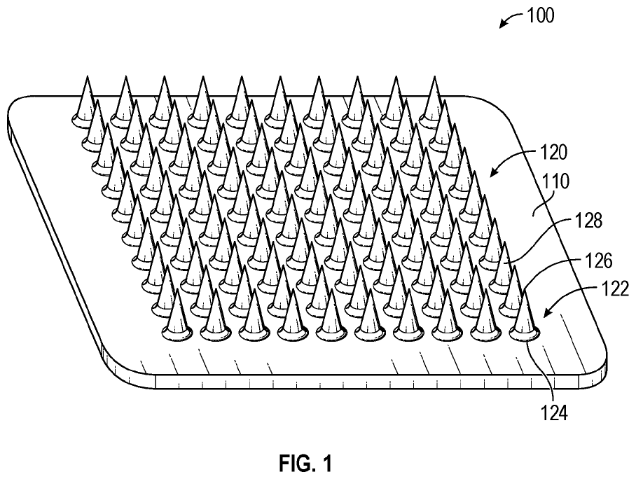

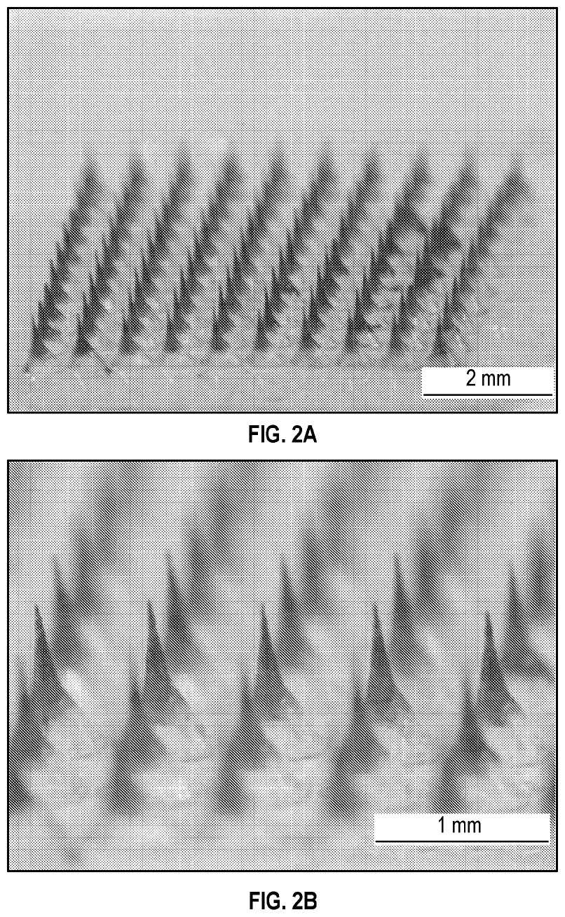

One example of a tattoo microneedle patch with an array of microneedles is depicted in FIG. 1. The microneedle patch 100 includes a base substrate 110 from which microneedles 120, in a 10×10 array, extend. The phrase “base substrate” and the term “substrate” or “backing” are used interchangeably herein. Each microneedle 120 has a proximal end 122 attached to the base substrate 110 directly, or indirectly, via one or more proximal portions 124, and a distal tip end 126 which is sharp and effective to penetrate biological tissue. The microneedle 120 has tapered sidewalls 128 between the proximal end 122 and distal end 126 of the microneedle 120. Other geometries are possible. Exemplary photographs of tattoo microneedle arrays as disclosed herein are shown in FIGS. 2A-2B.

The microneedles may be coated or uncoated. In some embodiments, the uncoated microneedle, or at least the distal tip portion of the microneedle, is fully water-soluble. In some other embodiments, the microneedle, or at least the distal tip portion of the microneedle, includes components that are water-soluble (e.g., a coating that includes the tattoo substance) and other components that are not water-soluble (e.g., an underlying microneedle structure on which the coating is disposed). For example, the underlying microneedle structure may be made of a metal, polymer, or silicon, that is coated with a water-soluble composition that includes the tattoo substance. This may be important because coated microneedles may generally be stronger (e.g., made of metal), which enables them to be thinner and can therefore be made with much higher density of microneedles per area of the microneedle patch. High resolution tattoo images may require a certain density of microneedles in the array for a microneedle patch, and the use coated microneedles may enable such densities and image resolution.

The microneedles of the microneedle patch typically are designed to insert into skin, but may be inserted into another suitable biological tissue.

In some embodiments, the length of the microneedle may be between about 50 μm and 2 mm, from about 100 μm to about 2000 μm, from about 100 μm to about 1500 μm, from about 200 μm to 1000 μm, or ideally between about 500 μm and 1000 μm. In some embodiments, the array of microneedles includes from 10 to 50,000 microneedles, from 25 to 10,000 microneedles, from 25 to 1000 microneedles, from 50 to 500 microneedles, or 100 microneedles (e.g., a 10-by-10 array of microneedles).

The microneedle patch may have a microneedle density in the array of from 10 microneedles/cm² to 20,000 microneedles/cm². In some embodiments, the area density may be from 50 microneedles/cm² to 5,000 microneedles/cm², or from 100 microneedles/cm² to 1000 microneedles/cm².

In some embodiments, the array of microneedles is configured to be inserted into a tissue area from about 0.5 cm² to about 10 cm².

The tattoo substance used in the microneedles of the microneedle patches described herein may be any material known in the art to be suitable for forming a tattoo in skin. For example, the tattoo substance may be a tattoo ink particle, or pigment particle, known in the art for use in conventional tattoos. It may be a single-color ink, multi-color inks, a changing-color ink, or a fluorescent ink (e.g., an ultraviolet (UV)-visible tattoo ink that emits visible light when exposed to ultraviolet (UV) light, such as blacklight tattoo ink). The tattoo substance can also be a dye, possibly associated with a particulate formulation, or any other material that is visible when administered into the skin.

In some embodiments, the microneedles, or at least the distal tip portions of the microneedles, include a mixture of a tattoo substance and a biocompatible, water-soluble polymer. Examples of such suitable water-soluble polymers include a poly(acrylic acid), polyvinylpyrrolidone, polyvinyl alcohol, and polysaccharides. However, essentially any suitable, biocompatible material that can provide the required mechanical properties needed to form a structure capable of being inserted into tissue and that subsequently is able to release the tattoo substance therein may be used.

In some preferred embodiments, the composition of the distal tip portion of the microneedle includes a tattoo substance and one or more excipient materials, wherein the tattoo substance is between 1 wt % and 50 wt % of the composition of the distal tip portion of the microneedle. In some embodiments, the tattoo substance is between 5 wt % and 40 wt %, or between 10 wt % and 30 wt %, of the composition of the distal tip portion of the microneedle.

In some embodiments, the microneedles include a hydrophobic matrix material in which a tattoo substance is dissolved or dispersed. In some embodiments, the microneedles include a proximal portion between the backing layer and the distal tip portions. The proximal portion may be substantially free of the tattoo substance. The proximal portion of the microneedles may include a water-soluble material, for example, polyvinylpyrrolidone, polyvinyl alcohol, a saccharide, such as sucrose, or a combination thereof.

In some embodiments, the backing layer (base substrate) of the microneedle patch includes tattoo substance. This tattoo substance may not be delivered into the skin, but may be present in the backing layer as a result of the manufacturing process. Microneedle patches used to administer drugs to the skin usually have drug only in the microneedles and not in the backing layer, because controlling the administered drug dose is important to medical treatment. Accordingly, it would be counter-intuitive to locate the substance that is intended to be delivered into the skin (e.g., the drug) in the backing layer of the microneedle patch. In contrast, a tattoo substance may be located in the backing layer even if it is not delivered into the skin, as long as tattoo substance in the distal tip portion of the microneedles is delivered into the skin. Such embodiments may be desirable for manufacturing the microneedle patch, for example using a single composition, e.g., a mixture of drug and water-soluble polymer, which might be used to mold the base substrate and microneedles in a single casting process.

In some embodiments, the microneedles within a given array of microneedles all contain the same tattoo substance. In other embodiments, the microneedles within a given array of microneedles may contain different tattoo substances. For example, the tattoo substances may be different in each microneedle, e.g., different colors, in different rows of microneedles, or sections/regions of the microneedle array, as needed to create a desired tattoo image.

The tattoo substance may be tattoo ink, or natural or synthetic pigments. It may be in particle form, or in liquid (e.g., solution, suspension or dispersion) or paste form (e.g., gel or cream) containing such particles. Such particles could be small enough to be cleared from the tissue for example by macrophages, e.g., with size of 10 nm to 20 um, or 100 nm to 10 um or 500 nm to 5 um, or as particles large enough to reduce clearance from the tissue, such as 10 um to 300 um or 20 um to 100 um or 30 um to 70 um.

The tattoo substance may be molecules that may diffuse away from the tissue (e.g., with rate of diffusion depending inversely on molecular weight) and/or may remain in the tissue, perhaps by binding to other substances or components in the tissue (e.g., bind to the tissue). The degree of binding could influence the duration that the tattoo remains in the tissue.

The tattoo substance may remain in the tissue essentially permanently, like conventional tattoos. The permanence could be enabled by the size of the pieces/particles of tattoo substance, by the interaction of the tattoo substance with other substances or components in the tissue or other factors.

The tattoo substance could remain in the tissue temporarily. The temporary nature of the tattoo substance remaining in the tissue could be enabled by methods known in the art for making temporary tattoos. This could be because the tattoo substance is cleared from the tissue in a form where the properties of the tattoo substance do not substantially change (e.g., clearance by diffusion, by macrophages) or could be due at least in part due to changes in the material properties, such as degradation of the tattoo substance by breaking chemical bonds in the tattoo substance (e.g., biodegradation), by dissolution of the tattoo substance (i.e., from a solid state to a dissolved state), by breaking up the tattoo substance into smaller pieces, or other mechanisms. For example, the tattoo substance may be henna, jagua, genipin or other materials known in the art to be used for temporary or semi-permanent tattoos.

The tattoo substance may remain in the tissue temporarily due to an intervention that makes the tattoo reversible. This could be accomplished by methods known in the art for tattoo removal, such as by using laser-based processes. The tattoo substance properties could be responsive to changes in environment (e.g., temperature, pH, tonicity) or could be responsive to energy input (e.g., laser, heat, ultrasound, light, electric field, magnetic field) to change properties of the tattoo substance that increase its rate of clearance from the tissue.

In some embodiments, the tattoo substance is encapsulated within or otherwise associated with particles that influence the duration that the tattoo remains in the tissue. This process could be at least in part mediated by diffusion of tattoo substance through the particles; binding of tattoo substance to the particles; and/or biodegradation, dissolution or other mechanisms by which the materials that make up the particles dissociate from the particles. The particles could be made of a biodegradable polymer such as poly(lactic acid), poly(glycolic acid), poly(lactic-co-glycolic acid) or poly(caprolactone), or mixtures thereof.

The microneedles including the tattoo substance may include other components that may also be delivered into the tissue or may be removed from the tissue after the tattoo substance is delivered. In some embodiments, the microneedle design may involve a microneedle core that does not contain the tattoo substance and a coating on part or all of the surface of the core that contains the tattoo substance. In this case, the coating on the microneedle mostly or completely remains in the tissue. The core may also remain in the tissue or may be mostly or completely removed. The microneedle design could alternatively involve a microneedle comprising a mixture of the tattoo substance and other component(s), or could be mostly or completely made of the tattoo substance. In this case, most or all of the microneedle remains in the tissue. The other components could dissolve in the tissue and be cleared from the tissue over a much shorter time scale compared to the possible clearance of the tattoo substance. In another scenario, the tattoo substance and some or all of the other components could remain in the tissue for approximately the same amount of time (i.e., including remaining in the tissue permanently).

The microneedle patch, may further include other structural elements (not shown in FIG. 1) that enhance storage and usability of the microneedles. For example, the patch may include a housing and/or other layers, such as a handle layer, on the side of the base substrate opposing the microneedles. Examples of such other additional structural components are described in U.S. Pat. No. 10,265,511 to McAllister et al., which is incorporated herein by reference.

### Methods of Making Microneedle Arrays

The arrays of microneedles described herein may be made by any suitable process. In a preferred embodiment, the arrays of microneedles are made using a molding process, which advantageously is highly scalable. The filling and molding steps described herein may be referred to herein as “casting”. In some embodiments, the casting methods, molds, and other equipment may be adapted from those known in the art, such as described in U.S. Pat. No. 10,828,478 to McAllister et al., which is incorporated herein by reference. The methods for making the microneedles preferably are performed under a minimum ISO 7 (class 10,000) process or an ISO 5 (class 100) process.

In some embodiments, the process includes preparing a composition with a tattoo substance dispersed, or dissolved, in an excipient, such as a water-soluble polymer, with or without an aqueous or organic solvent; filling a mold having a cavity defining a plurality of microneedles, e.g., an array of microneedles; and then solidifying the composition to form a plurality of microneedles. The solidification may involve drying to remove the solvent. In some embodiments, the process includes preparing a first liquid composition comprising a tattoo substance dispersed in an excipient material heated to a temperature greater than the melting point of the excipient material; casting the liquid composition in a mold having a cavity defining the microneedles; and then cooling the composition in the mold to a temperature that is less than the melting point of the excipient material to form the microneedles.

Centrifugation and/or vacuum may be used to aid these process, particularly in that the centrifugation and/or vacuum may help maintain microneedle shape as defined by the mold as the tattoo substance-excipient composition solidifies.

In some embodiments, a single-cast process is used to make the base substrate and the array of microneedles, as mentioned above. That is, the single cast of the tattoo substance and excipient forms not only a distal tip portion of the microneedles, but the proximal and any funnel portions of the microneedles along with the connected base substrate from which the array of microneedles extend.

In some embodiments, a two-cast or three-cast process is used. In these embodiments, the first cast of the tattoo substance and excipient forms only a distal tip portion of the microneedles. The subsequent cast may involve filling the remaining space of the mold on top of the distal tip portion of the microneedles to form a proximal portion of the microneedles. In some embodiments, a second cast is applied to the microneedle mold prior to removing the microneedles from the mold. In some embodiments, the second cast is of a different composition from the first cast. For example, the second cast may be aqueous-based, so that it could dissolve in vivo concurrently with the first cast composition. For example, aqueous-based casts may be made of a mixture of a polymer, such as polyvinyl alcohol (PVA) or polyvinylpyrrolidone (PVP), and a sugar, such as sucrose. In some preferred embodiments, the second cast is equal parts 18% PVP (360/55) and sucrose, such as 18 wt % PVP and 18 wt % sucrose. In some embodiments, the second cast is non-water-soluble, for example including a polymer such as poly(styrene) or poly(methylmethacrylate).

After the first cast composition is formed, it is transferred into a negative mold of the one or more microneedles, typically an array of between 10 and 1000 microneedles. This may be referred to as the “first cast.” The mold may then be centrifuged and/or vacuumed to facilitate displacement of any air bubbles and movement of the composition into the microneedle tips of the mold. The mold is then filled with a second composition that is suitable for forming the backing substrate and, optionally, a proximal end portion of the microneedles. In some embodiments, this second cast material may be or may include one or more water-soluble materials. After being filled, the mold may be centrifuged and/or vacuumed to facilitate and expedite the casting process.

The filled molds are permitted to solidify and/or dry as needed. The filled molds optionally may be dried at elevated temperatures and/or within a desiccant chamber to facilitate removal of excess water or moisture from the patches. The filled molds may be brought to a lower temperature after the filling process to cause a phase change of materials filled into the molds from liquid to solid. After the casts are substantially solidified and/or dry, the tattoo microneedle patch may be removed from the mold.

In some embodiments, the microneedle patches are manufactured using a three-cast process, where the first cast is a tattoo substance-matrix composition forming the distal tip portion of the microneedles, the second cast is an excipient forming a proximal end portion of the microneedles and/or a connector/funnel portion between the microneedles and the base substrate, and the third cast is a composition suitable for forming the base substrate. In these embodiments, the second cast excipients may dissolve upon insertion, and may serve as a separating layer between the distal tip portion (which may or may not dissolve in the skin) and the backing substrate (which may or may not dissolve in or on the skin). In some embodiments, it may be preferable to prevent the tattoo substance matrix composition and base composition from contacting one another, or mixing.

In some embodiments, making the microneedle arrays involves a coating process. For example, a microneedle array that does not comprise tattoo substance is made by molding, additive manufacturing, subtractive manufacturing, etching, 3D printing or other methods known in the art. Then, a coating is applied to the surfaces of the microneedles. The microneedles could be solid, or they could have invaginations, porosity, latticework, or other geometric features that provide surfaces for coating or cavities for filling that may be on the interior of the microneedle. A coating comprising tattoo substance is applied to the microneedles by dipping, spraying, electrodeposition or other methods known in the art for coating solid surfaces with liquid coatings. The coating is solidified by drying, phase change or other methods.

### Methods of Use

The microneedle arrays described herein may be used to deliver a tattoo substance into a tissue site in a human or other mammal, typically by applying a microneedle patch that includes an array of the microneedles to a skin surface in a manner to cause the microneedles to penetrate the stratum corneum and enter the viable epidermis and, possibly, the dermis. In some embodiments, delivery to the dermis is preferred.

As used herein, the phrase “penetrate a tissue surface,” includes penetrating a biological tissue surface with at least the distal tip ends of the microneedles. Upon separation of a microneedle from a substrate, a proximal end of a microneedle may be above a tissue surface, substantially level with a tissue surface, or below a tissue surface. The phrase “biological tissue,” generally includes any human or mammalian tissue. The biological tissue may be the skin or a mucosal tissue of a human or other mammal. It is envisioned that the present devices and methods may also be adapted to other biological tissues and other animals.

The microneedle patches may be self-administered or administered by another individual.

The methods described herein further include a simple and effective method of tattooing a person or animal with a microneedle patch. The method may include identifying an application site and, preferably, sanitizing the area prior to application of the microneedle patch (e.g., using an alcohol wipe). The microneedle patch then is applied to the person's skin/tissue and manually pressed into the patient's skin/tissue (e.g., using the thumb or finger) or using a device to facilitate patch application so that the microneedles penetrate the tissue surface. An applicator may be used that (i) does not contain any stored energy (i.e., the energy needed to press the microneedles into the tissue comes from the user), but guides the process of applying the patch to the tissue, or that (ii) contains stored energy (e.g., a compressed spring) that provides part or all of the force needed to press the microneedles into the tissue.

After delivery of the tattoo substance, the substrate (and remaining microneedle patch structure) may be removed from the patient's skin/tissue, at least in embodiments where the substrate remains intact.

In some embodiments, the microneedle patches described herein are used to deliver the tattoo substance into skin by inserting the microneedles across the stratum corneum (outer 10 to 20 microns of skin that is the barrier to transdermal transport) and into the viable epidermis and possibly the dermis. The small size of the microneedles enables them to cause little to no pain and target the intracutaneous space. The microneedles may dissolve once in the intracutaneous space to release the tattoo substance into the interstitial fluid and skin.

For microneedles that do not dissolve in the skin, the tattoo substance may be released accordingly to several mechanisms, such as diffusion, dissolution, and/or degradation. In embodiments, the microneedles may be manufactured such that the tattoo substance is released through an intact microneedle. In further embodiments, the microneedle may be formed of an excipient configured to degrade through, for example, hydrolysis, enzymatic activity, or other similar mechanisms. This allows for release of the tattoo substance at a rate that depends at least in part on the rate of degradation.

The information encoded by the tattoo substance may be pictorial or symbolic in nature (e.g., like a conventional tattoo for artistic or cosmetic purposes). It could be placed on any part of the body. It could be letters, words or characters (e.g., Chinese, hieroglyphics) in English or any other language. It could be numbers. It could be other symbols.

In some preferred embodiments, the tattoo substance is one that is useful as a cosmetic agent. For example, it could be permanent make-up, where the make-up may actually be permanent or may be temporary, but at least long lasting (e.g., longer than one day or one week or one month or longer). The tattoo substance could enhance or replace anatomical features, such as eyebrows, lips, hair (e.g., scalp micropigmentation), cheeks, eye lids (e.g., mascara, eye shadow) and other aspect of the face and head, as well as features on other parts of the body.

Cosmetic tattoos often cover large areas of skin and require customized artistry, but small tattoos with simple designs could be given quickly and painlessly by the microneedle patch. Self-administration may also be possible, and use of temporary tattoo ink may be especially suitable in these situations.

Medical tattoos can be very small and simple, such as those used to identify body sites for repeated treatments like radiotherapy or for veterinary tattoos identifying sterilization status of animals. A microneedle patch tattoo may be ideally suited in such scenarios. More complex, but relatively standardized designs like nipple-areola reconstruction therapy or dermatographic color correction of vitiligo may also be performed using the microneedle patch tattooing described herein, applied over a larger area with multiple patches and with customized coloring based on the patient's skin tone.

Many patients with chronic conditions wear medical alert bracelets, which in some cases could be replaced with tattoos. The tattoo image described herein could provide information on blood type or diabetes status, as well as other health care scenarios, including medical conditions (asthma, epilepsy), medications (blood thinner, allergies), medical devices (pacemaker), do-not-resuscitate orders, internet sites, and contact information.

The tattoo image described herein could be useful in public health, especially in settings where health care resources and medical recordkeeping are limited. Information about an important medical intervention could be recorded by a small, discreet tattoo. For example, a microneedle patch tattoo could be used to indicate the year or date on which a vaccination was administered, and this tattoo could be combined into a dual-function patch also used to administer the vaccine. This tattoo could serve as evidence of vaccination status to identify unvaccinated people during vaccination campaigns, e.g., for infectious disease control, elimination or eradication.

Microneedle patch tattoo images could also provide dynamic information in response to environmental changes. For example, the tattoos may be designed to respond to changes in light and/or heat. There also is a large body of literature on materials with properties that respond to a broad variety of physical and molecular inputs, such as glucose-responsive materials that change color or appearance to report glucose levels of interest to people with diabetes. Other materials are known to respond to change of pH, enzymes, binding reactions, redox state, ionic strength, mechanical stress or other stimuli that could be formulated in particle form and administered to the skin as a microneedle patch tattoo. Such an environmentally responsive tattoo could be a convenient, discreet method for patients to continuously monitor their health status.

Larger microneedle patch tattoos images can contain more information, either for direct readout of words, numbers or symbols, or for electronic readout, such as using a QR code that contains digitized information including a possible internet site link. While QR codes are standardized, other scanning codes could be used as well when combined with a suitable electronic reader.

In some embodiments, a microneedle patch may administer a tattoo substance into a tissue that encodes information that can be read from outside the body (i.e., a tattoo). Information can be in the form of a picture (e.g., an artistic or cosmetic tattoo with an image), letters/numbers, a pattern (e.g., QR code or RFID) or other arrangement of pieces of tattoo substance in the tissue, where the position and the properties (e.g., color, absorbance/emittance of electromagnetic, acoustic or other energy) of the pieces of tattoo substance encode the information.

Reading the information can be done visually by eye—either unaided or aided by an instrument that facilitates the eye seeing the tattoo substance—or done by a device without the involvement of the eye of the reader. The information that is being read could be in the form of visible light, for example in a pattern of color, intensity, spatial positioning and other characteristics. It could be non-visible light, such as ultraviolet or infrared light. The tattoo substance could be fluorescent so that when exposed to light (or energy from a non-light part of the electromagnetic spectrum) of a certain wavelength, it emits light (or energy from a non-light part of the electromagnetic spectrum) of a different wavelength, preferably one visible to the human eye.

The information could be from another part of the electromagnetic spectrum that is not considered light. The reading could be done by a detector of that part of the electromagnetic spectrum that is of interest, which can detect wavelength, intensity, spatial positioning and other characteristics of the tattoo substance in the tissue. The information could be acoustic in nature, using tattoo substances with acoustic properties different from the surrounding tissue. The information could be obtained from the tattoo substance in the tissue using an imaging device such as ultrasound, optical coherence tomography, x-ray, magnetic resonance imaging, x-ray computed tomography, positron emission tomography, radiography, elastography, photoacoustic imaging, near-infrared spectroscopy, magnetic particle imaging.

The tattoo image may be administered using a single microneedle patch or a collection of two or more patches that together provide the complete body of information, the complete picture, etc.

For example, the microneedle patch could be provided as part of a kit when applying more than one patch is desired. The kit may contain two or more microneedle patches and a template or alignment component that enables the user to position the microneedle patches relative to each other on the tissue surface. This would be useful when there is a need to use multiple patches that together make a composite tattoo. The microneedle patches may be applied to the same area of tissue (e.g., each patch has tattoo ink of a different color that together make an image with multiple colors or each patch has a certain number of microneedles, that each create a pixel of color in the tissue, and the use of multiple patches applies more microneedles that created more pixels to create an image with greater resolution (e.g., more pixels per area) or with more intensity (e.g., more ink per area).

The microneedle patches may together be applied to an area larger than the area of any individual patch. The total area could be less than, equal to or greater than the sum of the areas of the individual patches. For example, if the patches were placed with their edges immediately adjacent to each other, then the area could be roughly equal to the sum of the areas of the individual patches. If there is overlap of the placement of each individual patch, then the area could be less than the sum of the individual areas. If there is spacing between the edges of the patches, then the area could be greater than the sum of the individual areas.

The alignment component could be an article applied to the tissue and either left in place during application of the patches or removed before application of the patches. In the latter case, the alignment component may leave behind on the tissue surface a marking or impression or other guide for placement of the patches.

In some embodiments, the kit specifies a particular arrangement of the patches on the tissue. In some other embodiments, the kit may be adaptable to multiple arrangements according to how the user desires to use the patches, e.g., enabling users to customize the tattoo image according to their preferences.

In another embodiment, the kit may contain a collection of patches, each, for example, with one or more letters and/or numbers and/or symbols or other images. Combining two or more patches could create a collection of letters, numbers, symbols or other images that together form a particular tattoo image. Alternatively, the patches may be applied at locations distant from each other so that they do not together form a single tattoo image, but form two or more tattoo images. Patches from the same kit could also be applied to multiple different people, animals, etc. (e.g., matching tattoos to be shared by two people, or a set of tattoos to be applied to multiple animals for marking purposes). Any given patch in a kit would only be applied to one person.

In some embodiments, two or more microneedle patches may be provided as a kit without an alignment component. This would allow the user to design their own tattoo image using the patches of interest positioned in a pattern of interest on the tissue.

Microneedle patch designs can be customizable, where the customization can be performed at the time of manufacturing or afterwards (e.g., by the user). For example, a patch could have a collection of microneedles, some of which are functional (i.e., able to administer tattoo substances into tissue) and some of which are not functional. Microneedle patches could be manufactured with functional microneedles that are later converted into non-functional microneedles. That conversion could be done as part of the overall manufacturing process, or could be done by a user of the microneedle patch after manufacturing. A non-functional microneedle could be a site on the base substrate patch where there is no microneedle at all.

The customization could also be done by controlling the position and composition of the microneedles in a patch, where most or all of the microneedles contain tattoo substances. This customization could be done by making the patches this way, or could be done by making the patch with more microneedles on it, and subsequently removing certain microneedles. Removal of microneedles could be done as part of the overall manufacturing process, or could be done by a user of the microneedle patch after manufacturing.

A pattern can be encoded in a microneedle patch in multiple ways. A spatial pattern can be encoded by having a set of microneedles containing tattoo substances in that pattern on one or more patches, which is then transferred into the tissue. It is possible that there would be additional microneedles on the patch(es) that do not contain ink. In this way, there could be a standard array of microneedles on a patch that is customized by placing ink only into a fraction of the microneedles in order to achieve the pattern. The pattern could also encode different colors of ink or multiple types of tattoo substances with different properties (i.e., not just different colors). Each microneedle in an array could contain tattoo substances with the same or different properties, or contain no tattoo substances.

Customization could be designed by a user (i.e., not the manufacturer) and implemented by the manufacturer. For example, a user could provide a design to a manufacturer, and the manufacturer make the microneedle patch(es) according to that design. The design could be communicated to the manufacturer in a digitized format. Because microneedle patches are digital in nature (i.e., a microneedle patch transfers tattoo substances into tissue at a collection of specific locations-or pixels-that together form an image), a user could communicate the design by identifying the properties of each pixel in an array of pixels. Each pixel could have tattoo substances or no tattoo substances, and each pixel with tattoo substances could have specified properties (e.g., color). The instructions for each pixel can then be used as instructions for the design of each microneedle. Those instructions can be carried out by the manufacturer, who then provides the user with microneedle patch(es) according to the user's design, or could be carried out using an instrument (e.g., that selectively removes or disables microneedles) operated by the user or someone else at a site other than the site of microneedle patch manufacturing. In particular embodiments, the digitized design is transmitted from a user to a manufacturer via a web application or other computer or mobile device interface, using hardware and software adapted or known in the art.

In some cases, it may be desirable to have two or more microneedle patches that encode the same information. In other cases, it may be desirable to have microneedle patches that each encode unique information. These microneedle patches could be provided individually or as part of a multi-patch kit. The microneedle patches could contain tattoo substances as well as a drug, vaccine or other compound of medical, veterinary or other therapeutic, diagnostic or prophylactic interest (collectively referred to as “drug”). If part of a multi-patch kit, one or more patches could have tattoo substances and one or more other patches could have drug, or drug could be provided in the kit in a form that does not involve microneedles (e.g., drug in a tablet or formulated for injection). The information encoded in the microneedle patches could be about the drug, including the type of drug, information about its manufacturing (e.g., lot number, location, date), information about its time and/or location of use, information about the person receiving the drug, etc. The encoded information could link to a database or other source of information about the drug, manufacturing, time/location of use, person receiving the drug, etc.

Especially in animals, it may be desirable to apply the microneedle patches to tissue without hair to facilitate application of the microneedles into the tissue and/or to facilitate viewing or other access to the information encoded in the tissue (e.g., to see the tattoo). This could involve applying the microneedles to an area of tissue that naturally has little hair, such as the underside of the ear, the nose or certain areas on the underside of the animal (e.g., belly/groin). It could also involve an area of tissue that had had hair removed, either for the purpose of applying the microneedle patch or for some other purpose (e.g., as part of a surgical or other procedure, such as spaying, neutering, or other method of sterilization).

The information encoded in the tattoo image could be directly understood by a viewer of the tattoo (e.g., words, numbers, pictures). The information could be in the form of a code that can be understood by an algorithm, such as UPC codes, QR codes and RFID.

This invention can be further understood with reference to the following non-limiting examples.

### Example 1. Fabrication of a Tattoo Microneedle Patch Mold

A polydimethylsiloxane (PDMS) mold in the inverse of the desired shape of the microneedle patch was formed via laser cutting. First, a PDMS sheet was prepared by mixing two precursors of Sylgard 184 (Dow Corning, Midland, MI) at a ratio of 10:1. The mixture was degassed, poured into a flat-bottom container to a thickness of approximately 2.5 mm, and cured at room temperature (20° C. to 25° C.) for 2 days, and at 37° C. for an additional day.

The resulting solid PDMS sheet was drilled by a carbon dioxide (CO₂) cutting laser in vector mode in the VersaLaser engraver VLS 3.50 (Universal Laser Systems, Scottsdale, AZ) to generate an array of cone-like cavities to form microneedles in the mold. The microneedle dimensions and positions were controlled by drawing in AutoCAD software (Autodesk, San Rafael, CA) so that each microneedle had a base diameter of approximately 550 μm base diameter and a length of approximately 1.1 mm, where each microneedle was tapered to a tip with a radius of curvature of approximately 10 μm.

After the microneedle mold was formed, it was cleaned with a 20% (w/v) polyvinyl alcohol (PVA, 4-88, Millipore Sigma, Burlington, MA) solution in deionized water. This solution was applied to the drilled mold under vacuum and allowed to dry to form a PVA film at 40° C. overnight. Peeling off the PVA film removed burnt debris on the mold which had been generated during laser drilling.

### Example 2. Fabrication of a Tattoo Microneedle Patch

Tattoo microneedle patches were generally fabricated using a two-step molding process according to established methods, such as those described in Mistilis, M. J., Bommarius, A. S. & Prausnitz, M. R. Development of a thermostable microneedle patch for influenza vaccination. J. Pharm. Sci. 104, 740-749 (2015) and Edens, C., Collins, M. L, Goodson, J. L., Rota, P. A. & Prausnitz, M. R. A microneedle patch containing measles vaccine is immunogenic in non-human primates. Vaccine 33, 4712-4718 (2015).

Tattoo ink dry powder was dispersed at a concentration of 5% (w/v) in aqueous solution containing 10% (w/v) polyacrylic acid (PAA) (Polysciences, Warrington, PA) with the help of bath sonication. This dispersion was used as the first-case solution to fill the mold under vacuum for 20 minutes at room temperature (20° C. to 25° C.) to form the ink-laded microneedles. Excess liquid was removed from the mold surface after this step.

The second casting solution, consisting of 18% (w/v) polyvinyl alcohol (PVA) and 18% (w/v) sucrose (Sigma, St. Louis, MO), was then applied on the mold and dried under vacuum at room temperature (20° C. to 25° C.) for 3 hours to form the backing layer of the microneedle patch. The filled mold was further dried at 40° C. overnight before demolding the microneedle patch using adhesive tape (Scotch, 3M, St. Paul, MN). A microneedle patch formed according to this process is shown in FIGS. 2A and 2B.

### Example 3. Microneedles With Various Tattoo Inks

Different types of tattoo inks or pigments were loaded into microneedle patches to enable different appearance and function. The inks included (1) visible colored dry tattoo powders (Navy Blue TN15, Chinese Red TN31, and Indigo Black TN1, National Tattoo Supply, Allentown, PA), (2) UV-visible tattoo ink (Blacklight Invisible, Bloodline, Carson City, NV), and (3) thermochromic ink that changes color when heated about 31° C. (UniGlow, Tampa, FL). These inks were loaded in microneedle patches to get solid color microneedle patches, UV microneedle patches, and thermal microneedle patches, respectively.

To fabricate microneedle patches, dry ink powders were added directly to and suspended in casting solutions. For the liquid UV ink, the ink was centrifuged at approximately 5000×g for 3 minutes to separate the ink particles. The ink particles were then washed with an equal volume of deionized (DI) water and dried at 37° C. for two days to yield dry ink powder.

### Example 4. Application of Tattoo Microneedle Patch to Tattoo Skin

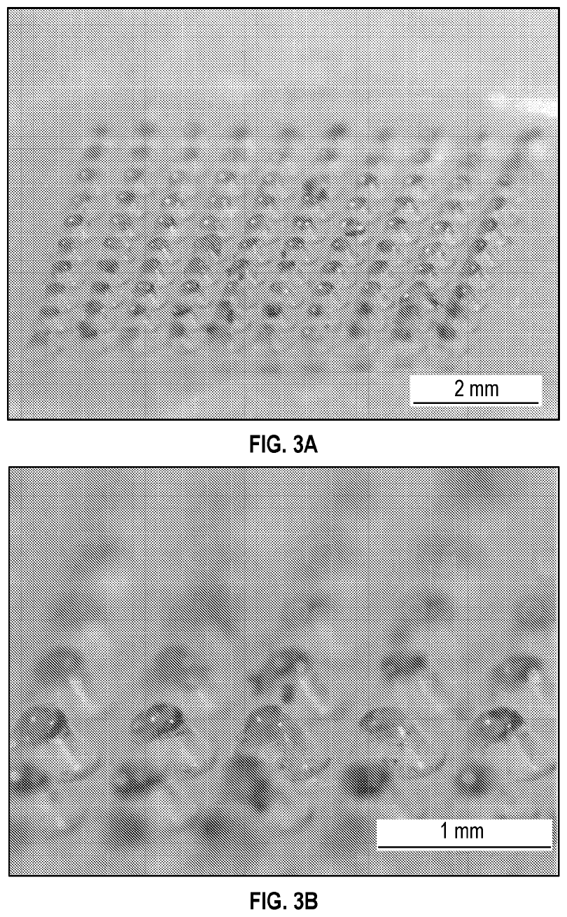

The feasibility of tattooing skin with microneedle patches was examined by applying tattoo microneedle patches to excised porcine skin ex vivo. Microneedle patches were briefly pressed against the skin for about 10 seconds, and left in place for about 15 minutes prior to peeling off. Ink deposition into the skin was accompanied by dissolution of the water-soluble microneedle, which left behind a used backing with no biohazardous sharps. For example, FIGS. 3A and 3B show the remaining backing and proximal portions of the tattoo microneedles after the distal tip portions dissolved to deposit ink into the skin. After application, the skin was gently cleaned by tissue paper (Kimwipe, Kimberly-Clark Professional, Roswell, GA) to wipe of residual ink and microneedle patch dissolution products from the skin surface. Photographs of the tattooed skin were taken by digital camera (Canon EOS 60D, Tokyo, Japan) from 20 cm or 1 m away.

### Example 5. Symbolic Tattoos by a Tattoo Microneedle Patch

Conventional microneedle patches for drug delivery are typically arranged as a square or circular array of microneedles. To create microneedle patches with other shapes that can communicate visual information, a CO₂ laser cutter was used to drill conical cavities in PDMS sheets to form molds with any desired pattern. In this way, microneedle patches were formed such that each microneedle behaved like a pixel or a dot that together created a tattoo shape or image. Each microneedle was made of a mixture of tattoo ink particles and a biocompatible, water-soluble polymer (e.g., poly(acrylic acid), PAA).

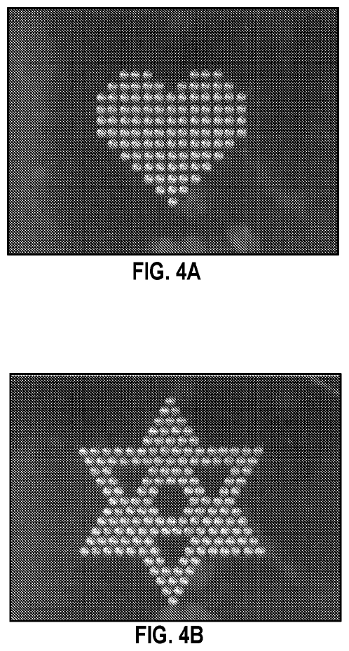

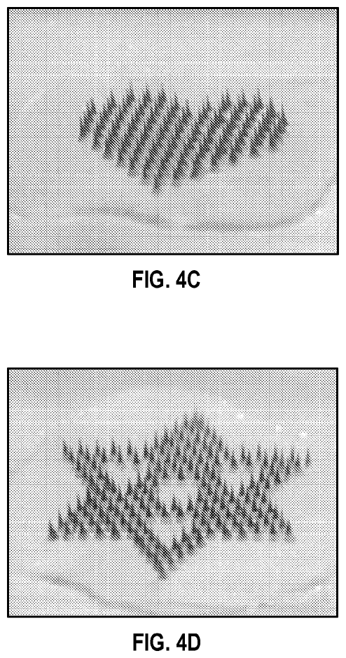

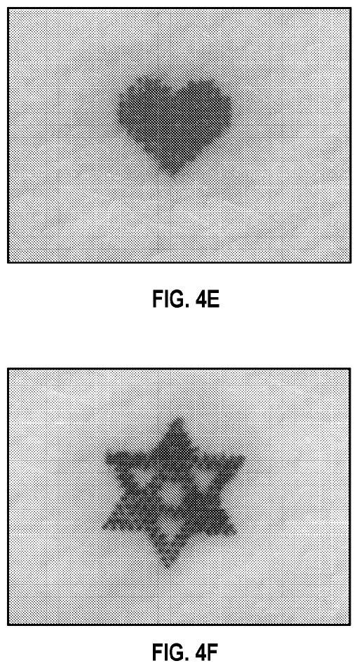

To demonstrate this approach, microneedle patch molds were prepared in the shape of a heart and a star, as shown in FIGS. 4A and 4B, respectively. These molds were used to fabricate microneedle patches having the same patterns, as shown in FIGS. 4C and 4D. When pressed into the skin, the tattoo ink was deposited by the microneedles into the skin to transfer the heart image (red in color) and star image (blue in color) as skin tattoos, as shown in FIGS. 4E and 4F, respectively.

### Example 6. Fabrication of a Tattoo Microneedle Patch for Vaccination

Two-component microneedle patches were designed to deliver inactivated poliovirus (IPV) vaccine and to make tattoo markers at the same time. Monovalent bulk IPV Type 2 (Middle East Forces (MEF) strain) was provided by Bilthoven Biologicals (Bilthoven, Netherlands). The antigen concentration in the bulk vaccine was measure to be 834 D-antigen units per milliliter (DU/ml). The stock IPV solution was concentrated with Amicon ultracentrifuge spin filters with 100 kDa molecular weight cutoff (Millipore Sigma). The final antigen concentration was determined to be 3.6 kDU/ml. All DU values were measured with sandwich enzyme-linked immunosorbent assay (ELISA), as described in Edens, C., Dybdahl-Sissoko, N. C., Weldon, W. C., Oberste, M. S. & Prausnitz, M. R. Inactivated polio vaccination using a microneedle patch is immunogenic in the rhesus macaque. Vaccine 33, 4683-4690 (2015).

The IPV vaccine and tattoo ink were loaded separately into different sections of a microneedle patch to obtain two-component microneedle patches. The tattoo ink was loaded into one side of the microneedle patch as described in Example 3. The IPV vaccine was similarly loaded into the other side of the microneedle patch, but with a different first-cast solution containing 3.6 kDU/ml IPV, 7.5 (w/v) maltodextrin (Sigma), and 2.5% (w/v) xylitol (Sigma), and a different second-cast solution containing 36% (w/v) maltodextrin, 12% (w/v) xylitol, and 1% (w/v) PVA. The two second-cast solutions contacted each other on the mold surface, and were dried into a single patch.

### Example 7. Application of a Microneedle Patch to Vaccinate and Tattoo Skin

Wistar rats (9 weeks old, female, Charles River Laboratories) were anesthetized via isoflurane inhalation during microneedle patch application. Five rats were immunized against IPV Type 2 with the two-component microneedle patch that administered 8 DPU IPV vaccine to the skin. The microneedle patches were pressed against shaved rat skin for approximately 10 seconds, and were left in place for about 15 minutes before peeling off. For the control group, six rats were immunized against IPV Type 2 by intramuscular injection in the thigh muscle of the hind limb. Intramuscular injection solutions were prepared by reconstituting and diluting the vaccine portion of a two-component microneedle patch to deliver the same dose in a 100 μl intramuscular injection.

Blood samples from the animals were collected over a six-week period to determine polio-specific neutralizing antibody titers. All blood samples were collected via tail-vein bleeding in collection tubes containing clot activators (BD Diagnostics, Franklin Lakes, NJ). Serum from the blood samples were separated via centrifugation at 6000×g for 1.5 minutes. Serum samples were stored in Eppendorf tubes at −20° C. until antibody titer analysis. All procedures in this study involving animals were approved by the IACUC at the Georgia Institute of Technology.

Polio-specific neutralizing antibody titers in serum samples were determined by the Division of Viral Diseases, National Center for immunization and Respiratory Diseases at Centers for Disease Control and Prevention (Atlanta, GA) using methods described in Combined immunization of infants with oral and inactivated poliovirus vaccines: results of a randomized trial in The Gambia, Oman, and Thailand. WHO Collaborative Study Group on Oral and Inactivated Poliovirus Vaccines. Bulletin of the World Health Organization 74, 253-268 (1996). Briefly, 80-100 cell culture infectious doses of 50% (CCID₅₀) of IPV Type 2 and serially diluted serum samples were mixed and incubated at 35° C. for 3 hours, followed by the addition of Hep-2 cells. After 5 days of incubation at 35° C. and 5% CO₂, the plates were stained with crystal violet, and the cell viability was determined by optical density measurements in a spectrophotometer. Then, neutralization titer was calculated via the Spearman-Karber method. The limit of detection for the assay was a 2.5 log₂ titer, and the precision of detection was ±0.5 log₂ titer.

The immune response following vaccination revealed that the neutralizing antibody response to vaccination was not significantly different in the microneedle patch and the intramuscular injection vaccination groups.

### Example 8. Tattoo Microneedle Patches for Medical Tattoos

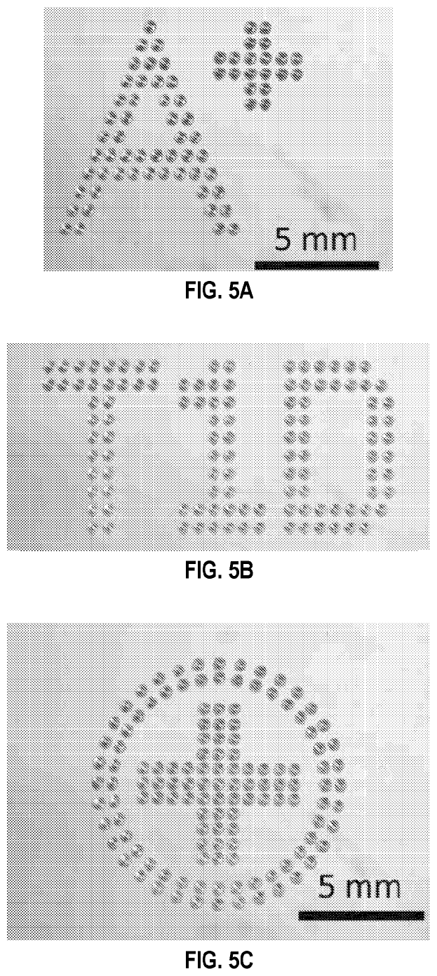

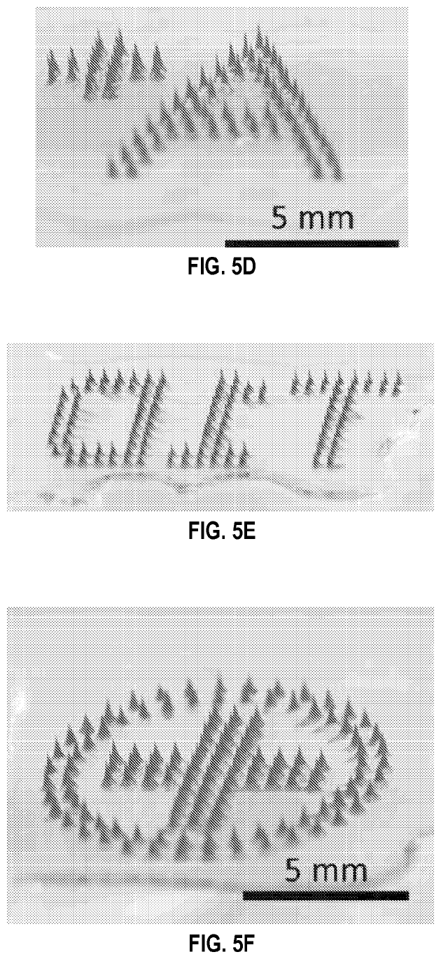

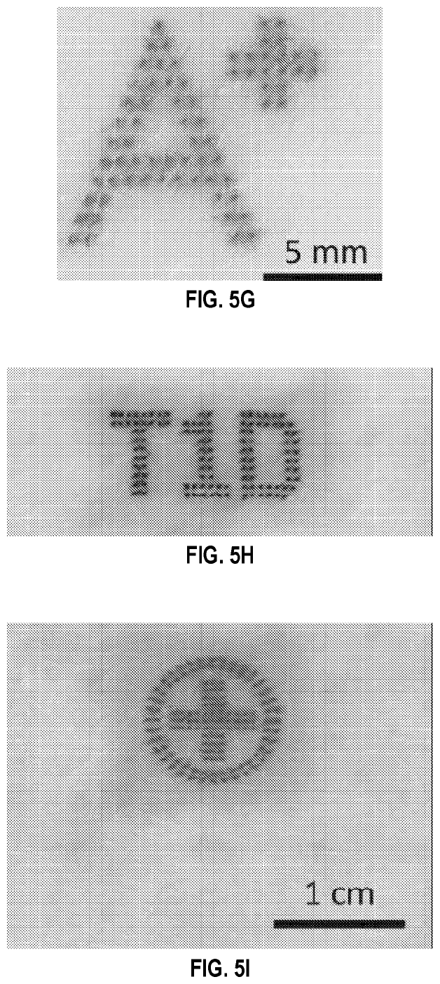

Tattoo microneedle patches were made for each of the eight different blood type codes and for a diabetes alert consisting of a “TID” label (i.e., type-1 diabetes) and a Red Cross symbol. As shown in FIGS. 5A-5C, the microneedle molds depict the tattoo images in the correct orientation. However, as shown in FIGS. 5D-5F, the tattoo microneedle patches display a mirror image of the tattoo so that the image is in the correct orientation when the tattoo is applied. These tattoos, as applied ex vivo to porcine skin, are shown in FIGS. 5G-5I.

These medical tattoos demonstrate the feasibility of developing tattoo microneedle patches to provide information using different numbers, letters, symbols, colors, and combinations thereof to create medical tattoo patches that can be easily administered and to create tattoo images that are easily read.

### Example 9. Light-Sensitive and Thermosensitive Tattoo Microneedle Patches

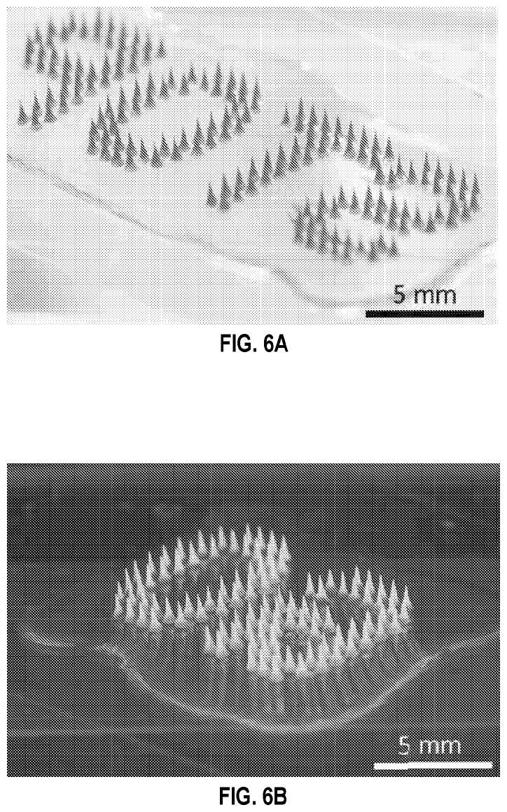

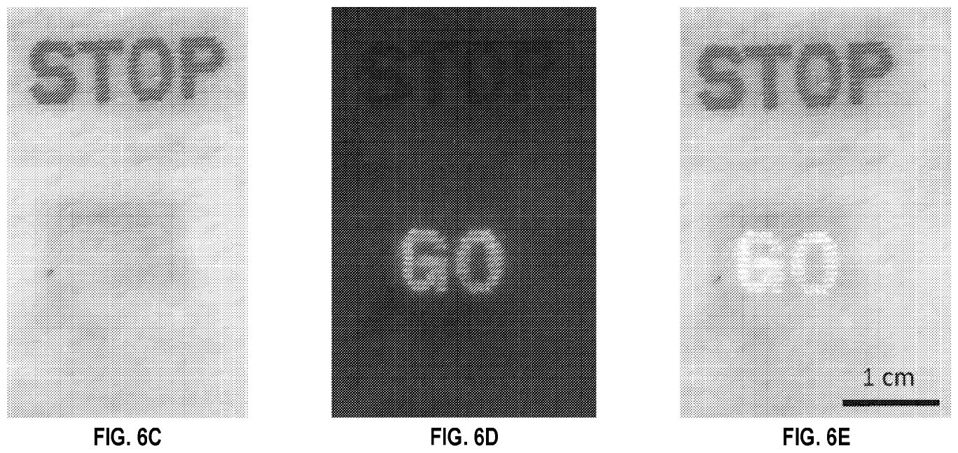

Since it may be useful to have tattoos that are responsive to environmental changes to provide different information in different settings, light-visible and UV-visible tattoo inks were combined in tattoo microneedle patches to create a tattoo with differential information based on the lighting conditions. These patches are shown in FIGS. 6A-6B, with the “STOP” patch of FIG. 6A being visible under daylight and the “GO” of FIG. 6B being visible under UV-light. In daylight, the “STOP” label was visible on porcine skin ex vivo, as shown in FIG. 6C, whereas the “GO” label was visible in darkness under UV-light, as shown in FIG. 6D. Notably, the “STOP” label was not visible in darkness, but as shown in FIG. 6E, both the “STOP” and “GO” labels were visible under UV-illumination in the daylight.

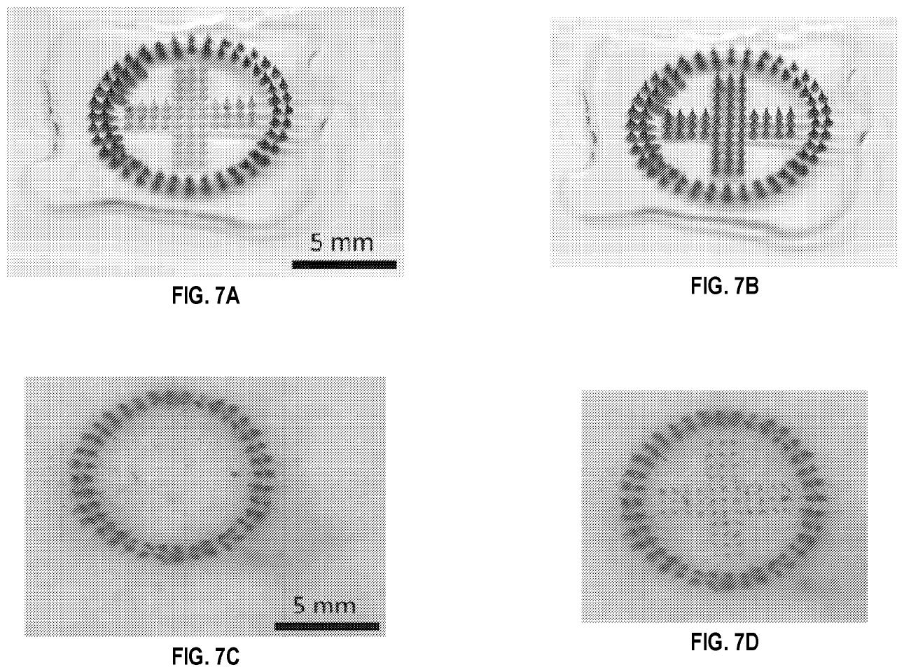

Two different inks were used to form a Red Cross symbol. The inner cross was made using a thermochromic ink that changes color in response to a temperature change, and the outer circle was made using a conventional tattoo ink. As shown in FIGS. 7A and 7C, the thermochromic ink is invisible or hardly visible in the ambient state. However, the inner cross changes color, reversibly, upon heating across a threshold of 40° C. both in the microneedle path and once the tattoo is applied ex vivo to porcine skin, as shown in FIGS. 6B and 6D, respectively. This type of thermosensitive tattoo may be used to monitor the temperature in the skin, such as during a fever or during a treatment involving exposure to elevated temperatures. Other thermochromic inks can change to different colors or at different temperatures, which could enable more color-change options for thermosensitive microneedle patch tattoos.

### Example 10. QR Code Tattoo Microneedle Patches

Tattoo microneedle patches may also be formed in the pattern of a QR code. A typically QR code consists of dark colored squares (called modules) on a white background. The grid pattern of the QR code stores digitized data, which can be text (e.g., with medical information) or internet links (e.g., to a database), and can be read by imaging devices such as smart phone cameras.

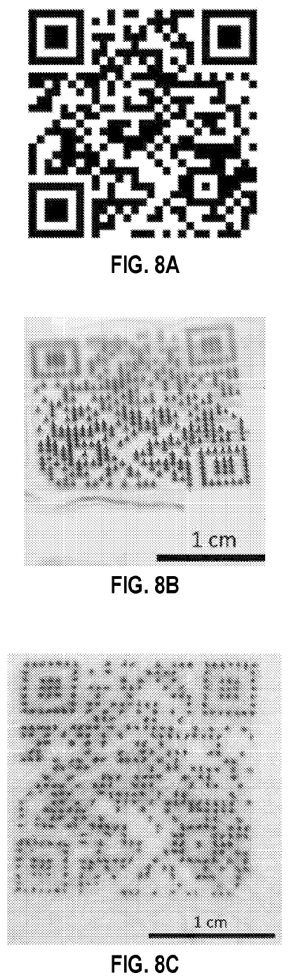

A target QR code, shown in FIG. 8A, had 29×29 modules that encoded a target URL address. The QR code microneedle patch, shown in FIG. 8B, was fabricated according to the methods disclosed herein and inserted ex vivo into porcine skin.

As shown in FIG. 8C, the tattooed dots were not square and were not immediately adjacent to each other, like with standard QR code modules. Despite these differences, as well as visual and morphological features of the skin, the tattooed QR code was successfully read by various well known applications, including WeChat (version 8.0.15, Tencent, Shenzhen, China) and TikTok (version 22.8.4, TikTok Pte., Singapore), as well as other widely installed code readers (i.e., code readers with more than 10,000,000 downloads) from the Google Play Store, such as QR & Barcode Scanner (version 2.2.18, Gamma Play, Hong Kong, China) and QR Scanner: Barcode Scanner & QR Code Scanner (version 2.4.5.GP, Simple Design, British Virgin Islands). All reading attempts tracked to the encoded URL address as expected.

Some embodiments of the present disclosure can be described in view of one or more of the following:

Embodiment 1. A microneedle patch for creating a tattoo image on skin, the microneedle patch comprising: a backing layer; and an array of microneedles extending from the backing layer, the microneedles each comprising a distal tip portion which comprises a tattoo substance; wherein the microneedles are configured to be inserted into a subject's skin, and wherein the microneedles are configured to release the tattoo substance in the skin, thereby creating the tattoo image in the skin, wherein the tattoo image is visible to the human eye.

Embodiment 2. The microneedle patch of Embodiment 1, wherein each microneedle in the array of microneedles corresponds to a dot, or a pixel, of the tattoo image or a portion thereof.

Embodiment 3. The microneedle patch of Embodiment 1 or 2, wherein the subject is a human.

Embodiment 4. The microneedle patch of Embodiment 1 or 2, wherein the subject is a domesticated animal, such as a pet or livestock.

Embodiment 5. The microneedle patch of any one of Embodiments 1 to 4, wherein at least a portion of the microneedles are configured to dissolve in vivo to release the tattoo substance.

Embodiment 6. The microneedle patch of any one of Embodiments 1 to 5, wherein the microneedles are configured to separate from the backing layer following insertion into the skin.

Embodiment 7. The microneedle patch of any one of Embodiments 1 to 6, wherein the microneedles further comprise a proximal portion between the backing layer and each of the distal tip portions, wherein the proximal portion is substantially free of the tattoo substance.

Embodiment 8. The microneedle patch of Embodiment 7, wherein the proximal portion comprises a water-soluble material, for example, polyvinylpyrrolidone, polyvinyl alcohol, a saccharide, such as sucrose, or a combination thereof.

Embodiment 9. The microneedle patch of any one of Embodiments 1 to 8, having a microneedle density in the array of from 50 microneedles/cm² to 20,000 microneedles/cm², such as from 90 microneedles/cm² to 1,000 microneedles/cm², or from 100 microneedles/cm² to 500 microneedles/cm².

Embodiment 10. The microneedle patch of any one of Embodiments 1 to 9, wherein the array of microneedles is configured to be inserted into a tissue area from about 0.5 cm² to about 10 cm².

Embodiment 11. The microneedle patch of any one of Embodiments 1 to 10, wherein the tattoo image is configured to be permanent.

Embodiment 12. The microneedle patch of any one of Embodiments 1 to 10, wherein the tattoo image is configured to be temporary.

Embodiment 13. The microneedle patch of any one of Embodiments 1 to 12, wherein the tattoo image comprises letters, numbers, symbols, patterns, and/or other encoded information that can be read from outside the body.

Embodiment 14. The microneedle patch of any one of Embodiments 1 to 13, wherein the tattoo image comprises a medical/veterinary alert and/or medical/veterinary medical information.

Embodiment 15. The microneedle patch of any one of Embodiments 1 to 14, wherein the tattoo substance comprises a single-color ink, multi-color inks, a changing-color ink, or a fluorescent ink.

Embodiment 16. The microneedle patch of any one of Embodiments 1 to 15, wherein the microneedles are drug-free.

Embodiment 17. The microneedle patch of any one of Embodiments 1 to 15, wherein the microneedles further comprise a drug.

Embodiment 18. A kit for creating a tattoo image comprising: two or more microneedle patches, each patch comprising: a backing layer; and an array of microneedles extending from the backing layer; wherein each microneedle of the array of microneedles comprises a distal tip portion comprising a tattoo substance, wherein the microneedles of each of the two or more microneedle patches are configured to be inserted into skin to form a tattoo image.

Embodiment 19. The kit of Embodiment 18, wherein the two or more microneedle patches are configured to cooperate to produce a composite tattoo resulting from the tattoo images from each microneedle patch being selectively positioned relative to one another, such as adjacent to one another and/or overlapping one another.

Embodiment 20. The kit of Embodiment 18 or19, further comprising an alignment template configured to facilitate selective positioning of the two or more microneedle patches on the skin.

Embodiment 21. The kit of any one of Embodiments 18 to 20, further comprising an applicator configured to facilitate inserting each of the two or more microneedle patches into the skin.

Embodiment 22. The kit of any one of Embodiments 18 to 21, wherein the tattoo image is visible to the human eye.

Embodiment 23. A method of applying a tattoo to skin, the method comprising: positioning a first microneedle patch onto a first area of a person's skin, wherein the first microneedle patch comprises a first array of microneedles, each of which comprises a distal tip portion comprising a tattoo substance; inserting at least the distal tip portion of each microneedle of the first array of microneedles into the person's skin; and releasing the tattoo substance in the person's skin to form a first tattoo image which is visible to the human eye.

Embodiment 24. The method of Embodiment 23, further comprising: positioning a second microneedle patch onto a second area of a person's skin, wherein the second microneedle patch comprises a second array of microneedles, each of which comprises a distal tip portion comprising a tattoo substance; inserting at least the distal tip portion of each microneedle of the second array of microneedles into the person's skin; and releasing the tattoo substance in the person's skin to form a second tattoo image which is visible to the human eye, wherein the first and second tattoo images cooperate to produce a composite tattoo resulting from the first and second tattoo images being selectively positioned relative to one another.

Embodiment 25. The method of Embodiment 24, wherein the first and second tattoo images are adjacent to one another or overlap one another.

Embodiment 26. The method of any one of Embodiments 23 to 25, wherein the microneedles dissolve in vivo to release the tattoo substance.

Embodiment 27. The method of any one of Embodiments 23 to 25, wherein the first and/or second microneedle patches are the microneedle patches of any one of Embodiments 1 to 17.

Embodiment 28. A method of making a tattoo microneedle patch, the method comprising: casting a first composition in a mold having a cavity defining an array of microneedles, wherein the first composition comprises a tattoo substance; and solidifying the first composition in the mold to form the array of microneedles, or at least a distal tip portion of the microneedles, in the mold, wherein each microneedle in the array of microneedles corresponds to a dot, or a pixel, of a predefined tattoo image or a portion thereof.

Embodiment 29. The method of Embodiment 28, further comprising: casting a second composition onto the distal tip portions of the microneedles to form proximal portions of the microneedles.

Embodiment 30. The method of Embodiment 29, wherein the second composition does not comprise a tattoo substance.

Embodiment 31. The method of any one of Embodiments 28 to 30, further comprising: casting a backing layer onto a base of the microneedles.

Embodiment 32. The method of any one of Embodiments 28 to 31, wherein at least the distal tip portion of each of the microneedles is configured to dissolve following insertion into a subject's skin.

Embodiment 33. The method of any one of Embodiments 28 to 32, wherein the predefined tattoo image or portion thereof is based on user-provided customization information.

Embodiment 34. The method of Embodiment 33, wherein the user-provided customization information comprises a digitized design in which a user has selected a characteristic and/or arrangement of each of the pixels that will correspond to each of the microneedles in the formed array of microneedles.

Embodiment 35. The method of Embodiment 33 or 34, wherein the user selects a tattoo substance color and/or a presence or absence of tattoo substance in each of the microneedles in the array of microneedles.

Embodiment 36. The method of any one of Embodiments 28 to 35, further comprising selectively removing one or more microneedles from the array of formed microneedles.

Embodiment 37. The method of any one of Embodiments 34 to 36, wherein the digitized design is transmitted from a user to a manufacturer via a web application or other computer or mobile device interface.

The term “about,” as used herein, indicates the value of a given quantity and can include quantities ranging within 10% of the stated value, or optionally, within 5% of the value, or in some embodiments, within 1% of the value.

While the disclosure has been described with reference to a number of exemplary embodiments, it would be understood by those skilled in the art that the disclosure is not limited to such disclosed embodiments. Rather, the disclosed embodiments can be modified to incorporate any number of variations, alterations, substitutions, or equivalent arrangements not described herein, but which are commensurate with the spirit and scope of the disclosure.

1. A microneedle patch for creating a tattoo image on skin, the microneedle patch comprising:
a backing layer; and
an array of microneedles extending from the backing layer, the microneedles each comprising a distal tip portion which comprises a tattoo substance;
wherein the microneedles are configured to be inserted into a subject's skin, and wherein the microneedles are configured to release the tattoo substance in the skin, thereby creating the tattoo image in the skin,
wherein the tattoo image is visible to the human eye.

2. The microneedle patch of claim 1, wherein each microneedle in the array of microneedles corresponds to a dot, or a pixel, of the tattoo image or a portion thereof.

3. The microneedle patch of claim 1, wherein the subject is a human.

4. The microneedle patch of claim 1, wherein the subject is a domesticated animal, such as a pet or livestock.

5. The microneedle patch of claim 1, wherein at least a portion of the microneedles are configured to dissolve in vivo to release the tattoo substance.

6. The microneedle patch of claim 1, wherein the microneedles are configured to separate from the backing layer following insertion into the skin.

7. The microneedle patch of claim 1, wherein the microneedles further comprise a proximal portion between the backing layer and each of the distal tip portions, wherein the proximal portion is substantially free of the tattoo substance.

8. The microneedle patch of claim 7, wherein the proximal portion comprises a water-soluble material, for example, polyvinylpyrrolidone, polyvinyl alcohol, a saccharide, such as sucrose, or a combination thereof.

9. The microneedle patch of claim 1, having a microneedle density in the array of from 50 microneedles/cm² to 20,000 microneedles/cm², such as from 90 microneedles/cm² to 1,000 microneedles/cm², or from 100 microneedles/cm² to 500 microneedles/cm².

10. The microneedle patch of claim 1, wherein the array of microneedles is configured to be inserted into a tissue area from about 0.5 cm² to about 10 cm².

11. The microneedle patch of claim 1, wherein the tattoo image is configured to be permanent.

12. The microneedle patch of claim 1, wherein the tattoo image is configured to be temporary.

13. The microneedle patch of claim 1, wherein the tattoo image comprises letters, numbers, symbols, patterns, and/or other encoded information that can be read from outside the body.

14. The microneedle patch of claim 1, wherein the tattoo image comprises a medical/veterinary alert and/or medical/veterinary medical information.

15. The microneedle patch of claim 1, wherein the tattoo substance comprises a single-color ink, multi-color inks, a changing-color ink, or a fluorescent ink.

16. The microneedle patch of claim 1, wherein the microneedles are drug-free.

17. The microneedle patch of claim 1, wherein the microneedles further comprise a drug.

18. A kit for creating a tattoo image comprising:
two or more microneedle patches, each patch comprising:
a backing layer; and
an array of microneedles extending from the backing layer;
wherein each microneedle of the array of microneedles comprises a distal tip portion comprising a tattoo substance,
wherein the microneedles of each of the two or more microneedle patches are configured to be inserted into skin to form a tattoo image.

19. The kit of claim 18, wherein the two or more microneedle patches are configured to cooperate to produce a composite tattoo resulting from the tattoo images from each microneedle patch being selectively positioned relative to one another, such as adjacent to one another and/or overlapping one another.

20. The kit of claim 19, further comprising an alignment template configured to facilitate selective positioning of the two or more microneedle patches on the skin.

21. The kit of claim 18, further comprising an applicator configured to facilitate inserting each of the two or more microneedle patches into the skin.

22. The kit of claim 18, wherein the tattoo image is visible to the human eye.

23. A method of applying a tattoo to skin, the method comprising:
positioning a first microneedle patch onto a first area of a person's skin, wherein the first microneedle patch comprises a first array of microneedles, each of which comprises a distal tip portion comprising a tattoo substance;
inserting at least the distal tip portion of each microneedle of the first array of microneedles into the person's skin; and
releasing the tattoo substance in the person's skin to form a first tattoo image which is visible to the human eye.

24. The method of claim 23, further comprising:
positioning a second microneedle patch onto a second area of a person's skin, wherein the second microneedle patch comprises a second array of microneedles, each of which comprises a distal tip portion comprising a tattoo substance;
inserting at least the distal tip portion of each microneedle of the second array of microneedles into the person's skin; and
releasing the tattoo substance in the person's skin to form a second tattoo image which is visible to the human eye,
wherein the first and second tattoo images cooperate to produce a composite tattoo resulting from the first and second tattoo images being selectively positioned relative to one another.

25. The method of claim 24, wherein the first and second tattoo images are adjacent to one another or overlap one another.

26. The method of claim 23, wherein the microneedles dissolve in vivo to release the tattoo substance.

27. A method of making a tattoo microneedle patch, the method comprising:
casting a first composition in a mold having a cavity defining an array of microneedles, wherein the first composition comprises a tattoo substance; and
solidifying the first composition in the mold to form the array of microneedles, or at least a distal tip portion of the microneedles, in the mold,
wherein each microneedle in the array of microneedles corresponds to a dot, or a pixel, of a predefined tattoo image or a portion thereof.

28-36. (canceled)
